(19) United States
(12) Patent Application Publication
(10) Pub. No.: US 2011/0010963 A1
(43) Pub. Date: Jan. 20, 2011

(54) FORM-FITTED GEL INSERT

(76) Inventor: Sue Webb, Alpharetta, GA (US)

Correspondence Address: Innovative IP, PLC, P.O. Box 217, New Smyrna Beach, FL, 32170, US

(21) Appl. No.: 12/553,793

(22) Filed: Sep. 3, 2009

Related U.S. Application Data
(60) Provisional application No. 61/225,382, filed on Jul. 14, 2009.

(51) Int. Cl. A43B 13/38; A61M 35/00

(52) U.S. Cl. 36/43; 604/289

(57) ABSTRACT

A form-fitted gel insert with a cushioned thickness that extends around and between the toes, below and on top of the front of the foot, wherein the device is generally yielding and resilient in response to impact, wherein toe movement necessary for effective sports play is not hampered, wherein pedicures remain protected from impact damage, wherein anti-bacterial features serve to reduce and/or prevent contamination, and wherein therapeutic ingredients beneficially impact nails and/or skin.

### CROSS-REFERENCE AND PRIORITY CLAIM TO RELATED APPLICATION

To the fullest extent permitted by law, the present U.S. Non-Provisional Patent Application claims priority to and the benefit of U.S. Provisional Patent Application entitled “Form-Fitted Gel Insert,” filed on Jul. 14, 2009, on behalf of inventor Sue Webb, and having been assigned Ser. No. 61/225,382.

### FIELD

The present disclosure relates generally to shoe inserts, and more particularly, to an insert for cushioning and protecting the front of the foot, in-between and around the toes, such as from uncomfortable and/or otherwise undesirable contact with the interior surface of the front of a shoe, and while providing a plurality of therapeutic benefits.

### BACKGROUND

A variety of shoe inserts have been suggested to serve a plurality of purposes. For example, several devices have been designed to function as a barrier, such as for preventing the spread of germs and/or disease when shoes are being tried for purchase. These devices can function effectively for their intended purpose, but because each is generally thin and only surrounds the exterior of the foot, each is disadvantageous in view of the present device.

Another form of toe-protecting device that has been described is a shield-style device for utilization in open toe shoes. These foot covers are disadvantageously stiff and unyielding. Such devices are generally intended to either protect the toe(s) from exposed impact or, if worn under a stocking, to prevent stocking wear.

Shoe inserts have also been directed at provision of insulative properties, whereby, for example, toe warmth during winter sports activities may be prolonged. In general, such devices have a thermal thickness that surrounds and at least partially covers and insulates the outer surface of the toe area of the foot. Such devices remain disadvantageous in view of the present disclosure.

Further devices have been disclosed, wherein toes are independently pocketed and/or subject to structural divide. However, these devices do not fit between AND around the toes, and do not provide for any gel structure formation, or realization of the benefits thereof. Further, none are adaptable for allowing toe movements necessary for accomplishment of sports activities.

Conversely, fluid-filled inserts have been described, such as for adjustable pressurization of an ankle and/or cushioning of the bottom of the foot; however, no such adaptation has been considered or anticipated for adaptation to individual toe placement and wear during sport play. Moreover, none protect the toenails from chipping or other damage that may be realized from undesirable contact and impact with the interior surface of the front of a shoe.

Therefore, it is readily apparent that there is a need for a form-fitted gel insert device that may conform to the front and top of the toe area, thereby avoiding the above-discussed disadvantages.

### BRIEF SUMMARY

Briefly described, in a preferred embodiment, the present device overcomes the above-mentioned disadvantages, and meets the recognized need for such a device, by providing a shoe insert for cushioning and protecting the front of the foot, in-between and around the toes, such as from uncomfortable and/or otherwise undesirable contact with the interior surface of the front of a shoe, and while providing a plurality of therapeutic benefits.

According to its major aspects and broadly stated, the present device is a form-fitted gel insert with a cushioned thickness that extends around and between the toes, below and on top of the front of the foot, wherein the device is generally yielding and resilient in response to impact, wherein toe movement necessary for effective sports play is not hampered, wherein pedicures remain protected from impact damage, wherein anti-bacterial features serve to reduce and/or prevent contamination, and wherein therapeutic ingredients beneficially impact nails and/or skin.

More specifically, the present device, according to the preferred embodiment, comprises a gel insert with a generally elongate divider with a first peripheral surface adapted to conform within the front interior of a shoe and with a plurality of toe insert compartments opposingly defined relative to the first peripheral surface, wherein each toe insert compartment is adapted with a generally cylindrical interior that is resilient, and essentially soft and meshy. The gel insert has a top and a bottom, according to the intended wear position within a shoe, wherein a generally flat treatment delivery zone is preferably defined on the top of the gel insert and proximate each of the plurality of toe insert compartments such that therapeutic additives may be delivered for nail treatment.

In another form, the gel insert is adapted with a base form of greater overall thickness, yet with a soft, yielding, and resilient nature, wherein a singular interior compartment is adapted with soft and flexible “teeth”, and wherein the interior compartment is adapted to receive all of a user's toes therewithin, such that the “teeth” generally serve as dividers therebetween.

In still another form, either as an alternate subcomponent of the preferred embodiment or as a therapeutic device for single toe application, a toe insert formed from gel may be shaped to essentially surround the entire toe, wherein a layer of charcoal may be embedded within the gel insert, and wherein the gel insert may be adapted with some measure of porosity in order to allow for moisture transfer therethrough, to be absorbed by the charcoal.

In addition and/or in lieu of the charcoal moisture control element, the alternate toe insert may be adapted with a therapeutic delivery system, proximate the base of the toe insert. In such an embodiment, vitamins or other beneficial treatment elements may be applied to the nail via insertion into the therapeutic delivery system.

In still another embodiment, one or more formed elements of the present device may be amenable to customization by the user. That is, the gel form of the insert may be susceptible to heating and/or other suitable manner of preparation that may allow a user to prepare the device to receive an imprint from the user's foot and/or toe(s) and to adaptively conform thereabout, retaining said conformation following removal of the foot and/or toe(s), and thereby enabling future accommodation of the particular user's foot and/or toe shape and configuration.

In yet still another embodiment, the gel insert may be of a more limited dimensional configuration, with a length to extend across the toes the wearer, and an extended width to extend from proximate the central region of each toe to the front of the toes, thereby permitting protective functionality to be retained, but without a base or bottom component, such that the gel insert is generally formed as a cap-type form. In such an embodiment, although some division between the toes is facilitated by the gel insert, full separation is not necessarily accomplished.

Therefore, a feature and advantage of the present device is its ability to protect the wearer's toes from uncomfortable and/or undesirable contact with the front interior surfaces of a shoe.

Another feature and advantage of the present device is its ability to cushion the wearer's toes proximate the top, front, side, and bottom surfaces thereof.

Yet another feature and advantage of the present device is its ability to prevent contact between the wearer's toes.

Still another feature and advantage of the present device is its ability to provide for moisture absorbance.

Yet still another feature and advantage of the present device is its ability to facilitate delivery of therapeutic treatment materials to the nail of a wearer.

Still yet another feature and advantage of the present device is its ability to conform to the particular individual anomalies of a wearer's foot and/or toe(s).

Still another feature and advantage of the present device is its ability to inhibit chipping of pedicure polish by providing a protective cushion about the toe nail.

Yet another feature and advantage of the present device is its ability to serve as a delivery mechanism for anti-microbial and/or anti-fungal medicaments.

Still yet another feature and advantage of the present device is its ability to facilitate shock absorption via a breathable structure.

Still another feature and advantage of the present device is its ability to provide a wearer with immediately enhanced comfort, stability, and control.

Yet another feature and advantage of the present device is its ability to protectively cushion the toes without inhibiting necessary movements for effective sports play.

Another feature and advantage of the present device is its beneficial anatomical shape, rendered from anti-fatigue material, wherein function is enhanced and greater bio-mechanical results are delivered.

Another feature and advantage of the present device is its washable nature and re-usable anti-microbial effectiveness.

These and other features and advantages of the present form-fitted gel insert device will become more apparent to one skilled in the art from the following description and claims when read in light of the accompanying drawings.

### BRIEF DESCRIPTION OF THE DRAWINGS

The present device will be better understood by reading the Detailed Description of the Preferred and Alternate Embodiments with reference to the accompanying drawing figures, in which like reference numerals denote similar structure and refer to like elements throughout, and in which:

FIG. 1 is a perspective, transparent view of a form-fitted gel insert, according to the preferred embodiment, showing the gel insert in position on a foot;

FIG. 2A is a cross-sectional view of a toe insert of the form-fitted gel insert of FIG. 1, according to the preferred embodiment of the present device;

FIG. 2B is a rear, perspective view of the form-fitted gel insert of FIG. 1, according to the preferred embodiment of the present device;

FIG. 3 is a partial, cut-away view of a toe insert of the form-fitted gel insert of FIG. 1, according to the preferred embodiment of the present device;

FIG. 4 is a bottom, partial cut-away and partial transparent view of the form-fitted gel insert of FIG. 1, according to the preferred embodiment of the present device, showing the device on the foot of a wearer;

FIG. 5A is a partial, cross-sectional view of a toe insert, according to an alternate embodiment of the present device, showing an impregnated desiccant;

FIG. 5B is a partial, cross-sectional view of a toe insert, according to an alternate embodiment of the present device, showing a therapeutic delivery system;

FIG. 6A is a rear, perspective view of a form-fitted gel insert, according to an alternate embodiment of the present device;

FIG. 6B is an bottom, partially transparent view of the form-fitted gel insert of FIG. 6A, showing the device on the foot of a wearer;

FIG. 6C is a partial, cutaway view of the interior surface of the form-fitted gel insert of FIG. 6A, showing a representative three-dimensional structure thereof;

FIG. 7 is a partially transparent, side view of a form-fitted gel insert, according to an alternate embodiment of the present device, showing the device on the foot of a wearer;

FIG. 8 is a perspective view of a form-fitted gel insert, according to the alternate embodiment shown in FIG. 7, showing the device worn on the foot of a wearer;

FIG. 9 is a front view of the form-fitted insert, according to the alternate embodiment shown in FIG. 7;

FIG. 10 is a bottom view of the form-fitted gel insert, according to the alternate embodiment shown in FIG. 7;

FIG. 11 is a side view of the form-fitted insert, according to the alternate embodiment shown in FIG. 7, showing the device worn on the foot of a wearer;

FIG. 12 is a top view of the form-fitted insert, according to the alternate embodiment shown in FIG. 7, showing the device worn on the foot of a wearer; and

FIG. 13 is a bottom of the form-fitted insert, according to the alternate embodiment shown in FIG. 7, showing the device worn on the foot of a wearer.

### DETAILED DESCRIPTION OF THE PREFERRED AND ALTERNATE EMBODIMENTS

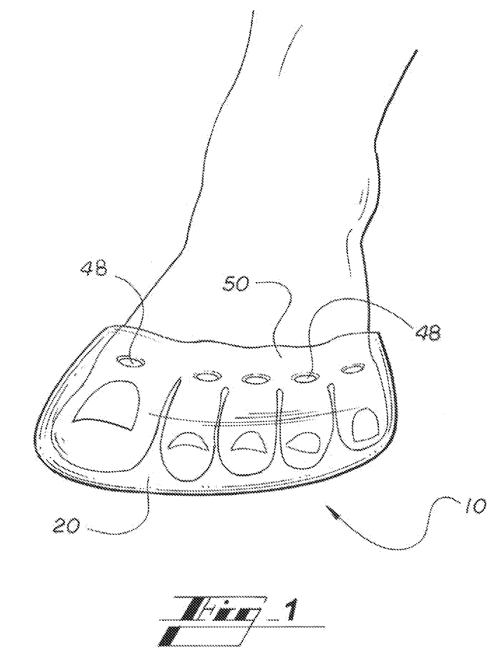

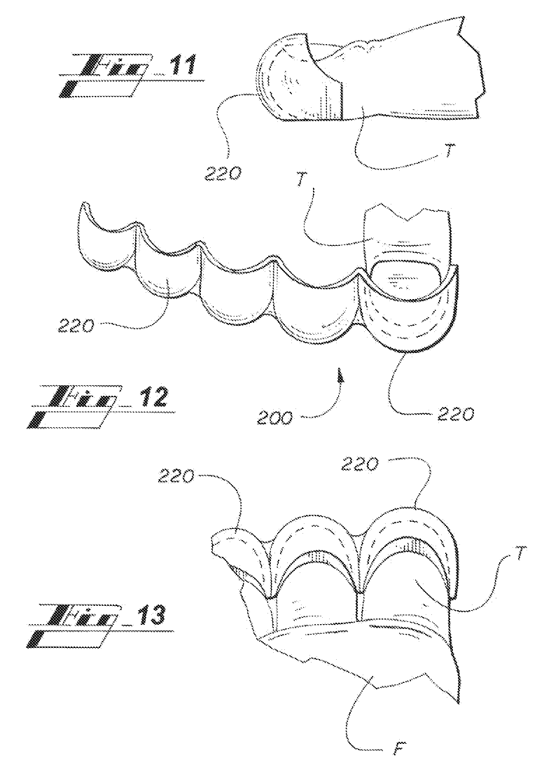

In describing the preferred and alternate embodiments of the present device, as illustrated in the FIGS. 1-13 and/or described herein, specific terminology is employed for the sake of clarity. The device, however, is not intended to be limited to the specific terminology so selected, and it is to be understood that each specific element includes all technical equivalents that operate in a similar manner to accomplish similar functions.

Referring now to FIGS. 1-4, the preferred embodiment is gel insert 10, comprising outer cushion region 20 and toe insert region 40, wherein gel insert 10 generally conforms to the front and top of the toe area, wherein outer cushion region 20 preferably cushions and protects the front of the foot F of a user, including toes T, and wherein toe insert region 40 preferably provides cushioning in-between and around a plurality of toes T. Preferably, gel insert 10 is formed from soft, pliable material and is preferably filled with cushioning gel to define a generally anatomically-shaped device that serves to enhance biomechanical performance of the user. Exemplary materials of manufacture and fill may include, without limitation, rubber, elastomer, silicone, collagen, polymer, and/or any suitable combination of materials, whether natural or man-made. The structure of gel insert 10 is preferably perforated in order to facilitate breathability, whereby undesirable buildup of heat may be minimized, while coincidently delivering anti-fatigue benefits. Additionally, the preferred materials of manufacture allow for true washability for gel insert 10, thereby enabling long-term, repeated usage.

Gel insert 10 is preferably of an elongate nature, wherein outer cushion region 20 defines first peripheral surface 22, and wherein first peripheral surface 22 is preferably shaped to conform to the front interior of a shoe. That is, first peripheral surface 22 is preferably generally convex; however, first peripheral surface 22 may define any generally formed yet conformable shape as is desirable to comfortably nest within the front of a shoe, without negatively influencing wearability and/or fit, yet while providing a protective and cushioning barrier for the front of the foot F and toes T. Outer cushion region 20 is therefore preferably gel-filled, and preferably opposingly and integrally related to toe insert region 40.

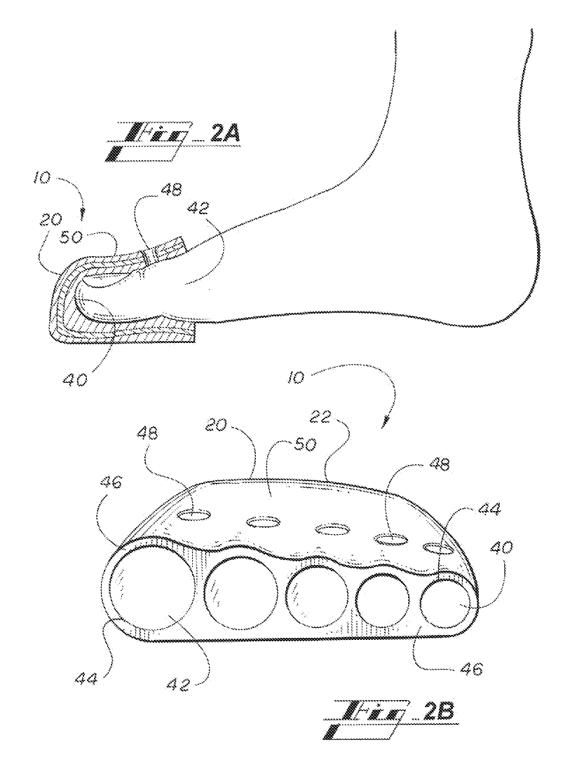

According to the preferred embodiment, toe insert region 40 is adapted with plurality of toe insert compartments 42, wherein each toe insert compartment 42 preferably defines generally cylindrical interior space 44 for receiving a toe, T, therewithin. As may best be seen in FIG. 2A, each toe insert compartment 42 is preferably formed with resilient band-like features 46 that serve to impart a snugly secure friction fit about each toe T. While toe insert region 40 is preferably gel-filled, as are the remaining regions of gel insert 10, it is desirable for each cylindrical interior space 44 of each toe insert compartment 42 to be soft and meshy, essentially surrounding each toe T in a supportively comfortable manner, yet without restricting necessary movements, such as that required for serious sports participation and play. According to this preferred embodiment, gel insert 10 serves to cushion a wearer's toes proximate the top, front, side, and bottom surfaces thereof, minimizing chipping of pedicure polish, preventing uncomfortable pushing of the toe nails and toes against the interior surface of a shoe, such as during tennis play, and preventing undesirable contact between the wearer's toes. Moreover, the preferred nature of interior space 44 facilitates comfortable accommodation of particular individual anomalies, such as crooked toes or the like.

Preferably, toe insert region 40 is adapted with a plurality of treatment delivery ports 48, exemplarily shown in FIGS. 1-2B, each preferably defined proximate upper surface 50 of gel insert 10, wherein therapeutic additives may be delivered therethrough, such as for nail treatment. Exemplary therapeutic additives, without limitation, may include anti-microbial treatments, anti-fungal medicaments, vitamins, minerals, aromatics, oils, and/or any other desired additive for delivery to the toe T, and particularly, the nail.

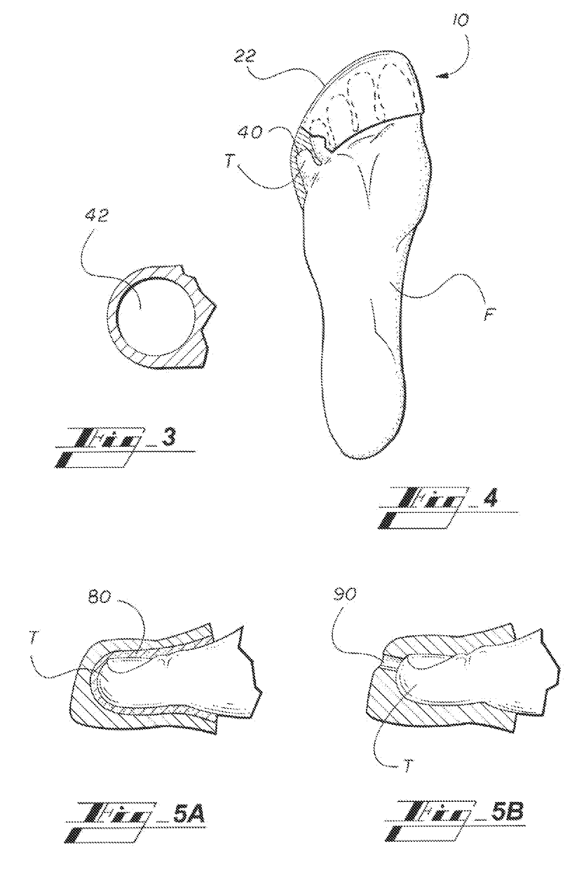

Referring now to FIGS. 5A and 5B, further exemplary embodiments are shown, wherein gel insert 10 may be impregnated with desiccant 80 and/or may be adapted with alternate therapeutic delivery system 90. It should be appreciated that either or both of these features may be incorporated in the preferred embodiment, as described, or may be adapted for inclusion in further alternate embodiments, such as embodiments comprising fewer than five toe insert compartments 42. That is, it is anticipated that, for example, an alternate form of gel insert 10 may be formed with outer cushion region 20 and only a single toe insert compartment 42, such as for the big toe; or, another alternate embodiment of gel insert 10 may be formed with outer cushion region 20 and only two toe insert compartments 42, such as for the big toe and the smallest toe; and so on.

Nonetheless, irrespective of the total number of toe insert compartments, impregnated desiccant 80 may be defined as a layer, or layers of charcoal embedded within gel insert 10. It should be apparent to one skilled in the art that any suitable desiccant material may be selected, although charcoal is preferred. The preferred perforated structure for gel insert 10 facilitates transfer of moisture, generally wicked from the surface of the toe T to the desiccant 80. This feature serves to enhance temperate comfort, as well as to prevent the disadvantageous establishment of an environment suited for bacterial growth.

Alternate therapeutic delivery system 90, such as depicted in FIG. 5B, allows for specifically directed nail therapy. As with the plurality of treatment delivery ports 48, therapeutic delivery system 90 is defined proximate upper surface 50 of gel insert 10. However, therapeutic delivery system 90 is more particularly positioned proximate the nail region of a toe held within gel insert 10, rather than plurality of treatment delivery ports 48, which may serve to deliver therapeutics to the generalized toe itself, for example, including skin treatments. Again, exemplary therapeutic additives, without limitation, may include anti-microbial treatments, anti-fungal medicaments, vitamins, minerals, aromatics, oils, and/or any other desired additive for delivery to the toe T, and particularly, the nail.

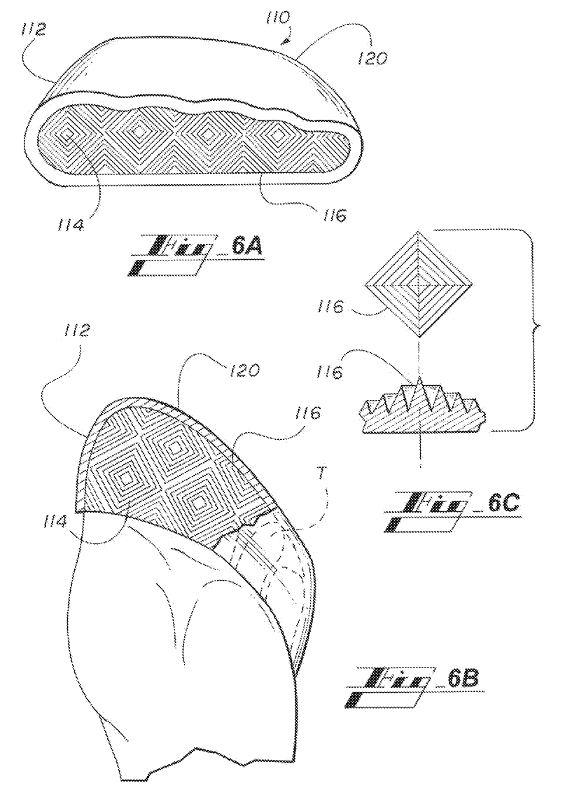

In still another alternate embodiment, referring now to FIGS. 6A-6C, gel insert 110 is adapted as base form 112 of greater overall thickness, yet with a soft, yielding, and resilient nature, wherein singular interior compartment 114 is adapted with plurality of soft and flexible dividers 116, and wherein interior compartment 114 is adapted to receive all of a user's toes therewithin, such that dividers 116 function to comfortably space and cushion between the toes T. Dividers 116, as shown in FIG. 6C, may be defined by an overall diamond-shape configuration, with the central diamond having a greater height, and diminishing outwardly therefrom, in order to facilitate comfortable spacing and cushioning between the toes. Of course, one skilled in the art would readily recognize that other shapes and configurations could be utilized without departing from the intended scope of the present disclosure. According to this alternate embodiment, outer cushion region 120 remains similarly formed as with the preferred embodiment, to cushion and protect the front of the foot F of a user, including toes T.

In yet still another embodiment, gel insert 10 may be amenable to customization by the user. That is, gel insert 10 may be formed from materials susceptible to generally permanent deformation, such as following heating, boiling, microwave exposure, and/or other suitable manner of preparation, wherein a user may prepare device 10 to receive an imprint from the user's foot F and/or toe(s) T and to adaptively conform thereabout, retaining said conformation following removal of the foot F and/or toe(s) T. and thereby enabling future accommodation of the particular user's foot and/or toe shape and configuration.

Still, in a further alternate embodiment, it is considered that gel insert 10 could be formedly related to a sock (not shown) and/or a partial sock, such as a footlet (also not shown). In this embodiment, gel insert 10 could be essentially affixed within the interior of a sock, wherein upon application of the sock to the foot of the wearer, gel insert 10 would receive the foot and toes of the user, cushioning same from within the sock structure.

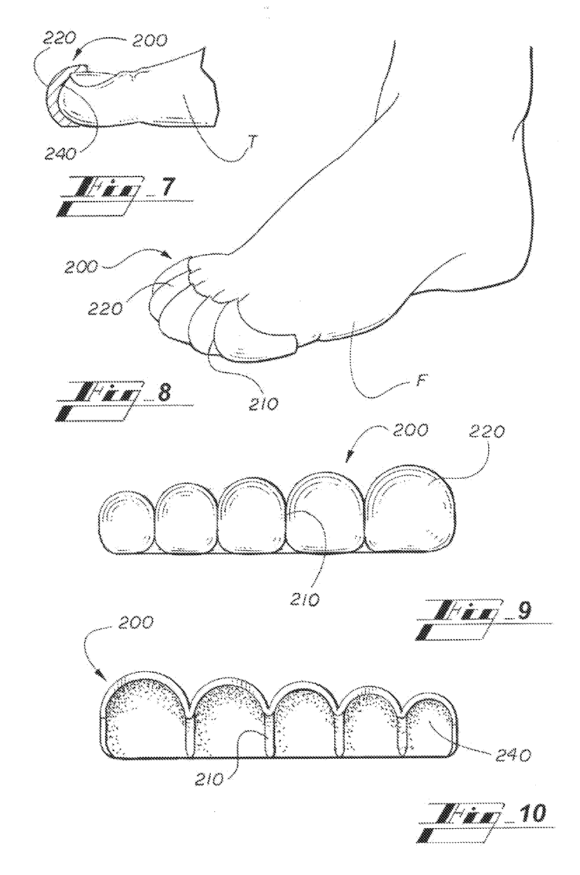

Finally, in yet still another embodiment, with particular reference to FIGS. 7-13, gel insert 200 may be of a more limited dimensional configuration, with a length to extend across the toes the wearer, such as depicted in FIG. 8, and an extended width to extend from proximate the central region of each toe to the front of the toes, such as depicted in FIG. 7, thereby permitting protective functionality to be retained, but without a base or bottom component, such that gel insert 200 is generally formed as a cap-type form. In such an embodiment, although some division between the toes is facilitated by related segments 210 of gel insert 200, full separation is not necessarily accomplished.

Outer cushion region 220 generally abuts the interior of a shoe and/or sock of a wearer, as desired, and toe insert region 240 is less fully formed, such that the entire toe of the wearer is not surrounded, but gel insert 200 generally conforms to the front and top of the toe area, outer cushion region 220 cushions and protects the front of the foot F of a user, including toes T, and wherein toe insert region 240 provides general cushioning in-between a plurality of toes T.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

I claim:

1. A gel-filled insert, comprising:
an outer cushion region, and
a toe insert region,
wherein said gel-filled insert generally conforms to a front and a top of a wearer's toe area, wherein said outer cushion region is a barrier between a front of a wearer's foot and one or more toes and a front interior of a wearer's shoe or sock, and wherein said toe insert region extends at least partially in-between the wearer's toes.

2. The gel-filled insert of claim 1, wherein said toe insert region extends in-between and around the wearer's toes.

3. The gel-filled insert of claim 1, wherein said gel-filled insert further comprises a generally elongate divider with a first peripheral surface defining said outer cushion region, wherein said toe insert region further comprises a plurality of toe insert compartments opposingly defined relative to said first peripheral surface, and wherein each said toe insert compartment is adapted with a generally cylindrical interior space.

4. The gel-filled insert of claim 3, wherein said gel insert has a top and a bottom, according to an intended wear position within a shoe, wherein a generally flat treatment delivery zone is defined on said top of said gel insert, with a plurality of treatment delivery ports defined therein, proximate said plurality of toe insert compartments.

5. The gel-filled insert of claim 1, wherein said toe insert region is defined as a singular compartment, and wherein said singular compartment is adapted with a non-smooth interior surface.

6. The gel-filled insert of claim 1, further comprising a layer of desiccant within said gel insert.

7. The gel-filled insert of claim 1, wherein said toe insert region is adapted to receive fewer than five toes therewithin, and wherein said outer cushion region generally surrounds the fewer than five toes.

8. The gel-filled insert of claim 1, wherein said gel-filled insert is adapted with at least minimal porosity.

9. The gel-filled insert of claim 1, further comprising a therapeutic delivery system defined proximate a base of said toe insert.

10. The gel-filled insert of claim 1, wherein said gel-filled insert is comprised of material suitable for preparation and retention of an imprint conformation of at least a portion of the foot of the wearer.

11. The gel-filled insert of claim 1, wherein said outer cushion region is dimensioned to extend across an upper and a frontal surface of the toes of the wearer, wherein said toe insert region is dimensioned to receive the upper and the frontal surface of the toes of the wearer, and wherein a plurality of division structures extend at least minimally between the toes of the wearer.

12. The gel-filled insert of claim 1, wherein said gel-filled insert is manufactured and filled with one ore more materials selected from the group consisting of rubber, elastomer, silicone, collagen, and polymer.

13. The gel-filled insert of claim 3, wherein each said toe insert compartment further comprises resilient band-like elements.

14. The gel-filled insert of claim 4, wherein said plurality of treatment delivery ports are adapted to deliver one or more therapeutic additives therethrough, and wherein said therapeutic additives are selected from the group consisting of anti-microbial treatments, anti-fungal medicaments, vitamins, minerals, aromatics, and oils.

15. The gel-filled insert of claim 6, wherein said desiccant is charcoal.

16. The gel-filled insert of claim 5, wherein said non-smooth interior surface further comprises a plurality of soft and flexible dividers, and wherein each said divider defines a three-dimensional, diamond-shape structure.

17. The gel-filled insert of claim 1, further comprising at least a portion of a sock, said at least a portion of said sock formedly related to said gel-filled insert.

18. The gel-filled insert of claim 1, wherein said gel-filled insert defines a cap-type form without a base.

19. A gel-filled insert, comprising:
a perforated outer cushion region, and
a toe insert region having a plurality of separated toe insert compartments, each said compartment surrounding a toe,
wherein said gel-filled insert generally conforms to a front and a top of a wearer's toe area, wherein said outer cushion region is a barrier between a front of a wearer's foot and one or more toes and a front interior of a wearer's shoe or sock, and wherein said gel-filled insert is further adapted with a moisture control medium.

20. A method for delivery of therapeutic medicaments to a region of a toe, comprising the steps of:
obtaining a gel-filled member with an outer surface and one or more inner, toe-receiving compartments, wherein said gel-filled member is adapted with one or more apertures proximate said one or more inner, toe-receiving compartments;
placing said gel-filled member into position relative to the toes of a wearer;
placing a therapeutic medicament into said one or more apertures; and,
allowing the therapeutic medicament to be absorbed by a region of the toe proximate said one or more apertures.
